(19) United States
(12) Patent Application Publication
(10) Pub. No.: US 2024/0335481 A1
(43) Pub. Date: Oct. 10, 2024

(54) BLAUTIA SP. STRAIN, LEUCONOSTOC SP. STRAIN, OR RUMINOCOCCUS SP. STRAIN AND ENDOPLASMIC RETICULUM DERIVED THEREFROM, AND ANTI-INFLAMMATORY AND ANTIBACTERIAL USES THEREOF

(71) Applicant: BIOBANKHEALING INC, Seongnam-si Gyeonggi-do (KR)

(72) Inventors: Ki Sung KANG, Yongin-si Gyeonggi-do (KR); Dong Ho LEE, Seongnam-si Gyeonggi-do (KR); Won Suk LEE, Seongnam-si Gyeonggi-do (KR)

(73) Assignee: BIOBANKHEALING INC, Seongnam-si Gyeonggi-do (KR)

(21) Appl. No.: 18/294,819

(22) PCT Filed: Aug. 2, 2022
(86) PCT No.: PCT/KR2022/011406

§ 371 Date: Apr. 1, 2024

(30) Foreign Application Priority Data
Aug. 2, 2021 (KR) 10-2021-0101636
Aug. 2, 2021 (KR) 10-2021-0101638
Aug. 2, 2021 (KR) 10-2021-0101640
Aug. 2, 2021 (KR) 10-2021-0101641
Aug. 2, 2021 (KR) 10-2021-0101642

(51) Int. Cl. A61K 35/74; A23L 33/135; A61P 29/00; A61P 31/04

(52) U.S. Cl. CPC A61K 35/74; A23L 33/135; A61P 29/00; A61P 31/04

(57) ABSTRACT

The present disclosure relates to a novel microorganism, a lysate thereof, a culture solution thereof, an extract of the culture solution, a vesicle derived from the novel microorganism, and anti-inflammatory and/or antibacterial use of the foregoing. The novel strain and the vesicle derived therefrom according to an embodiment may be useful for the prevention, amelioration, or treatment of inflammation-related conditions or bacterial infections.

### TECHNICAL FIELD

The present disclosure relates to a novel microorganism, a lysate thereof, a culture solution thereof, an extract of the culture solution, a vesicle of the novel microorganism, and inflammatory and/or antibacterial use of the foregoing.

### BACKGROUND ART

A microbiome refers to microorganisms in a given environment and the entire genetic information thereof, and is a collection of genomes that represent the entire genetic information of a single organism. In this regard, the human microbiome refers to microorganisms living inside and outside the human body and the entire genetic information thereof.

The human body lives in a symbiotic relationship with many microorganisms, and in particular, the intestines are optimal environments for microorganisms to consume nutrients and form systematic communities, and thus there are the largest population of microorganisms in the intestines. The intestinal microorganisms supply nutrients that cannot be produced by enzymes of a host, and are deeply associated with the metabolism and immune system of a host, but have been also reported to be related to the occurrence of various diseases such as irritable bowel syndrome, obesity, atopy, depression, rheumatoid arthritis, autism spectrum disorder, dementia, and the like.

Recently, Western dietary habits and indiscriminate use of antibiotics cause an imbalance in the intestinal microbiota, leading to poor intestinal health. Also, research on the intestinal microorganisms and various diseases related thereto has highlighted the importance and interest in the intestinal microorganisms.

Meanwhile, a vesicle is a nano-sized substance of about 20 nm to about 200 nm produced and released by cells, and can move freely between cells. In addition, such a vesicle contains membrane lipids, membrane proteins, DNA or RNA, etc., and these genetic materials are known to act as a complex to transfer cells and toxic factors between cells and play a role in regulating inflammation and immune responses. From unicellular to multicellular organisms, information exchange between cells is an essential process in life phenomena, and vesicles have been recently recognized as a mediator of exchanging information between cells. Thus, methods of using vesicles as a drug carrier are being developed.

Accordingly, there is a need to develop substances for ameliorating, preventing, or treating diseases by using novel microorganisms derived from the human intestines and vesicles derived therefrom.

### DETAILED DESCRIPTION OF THE DISCLOSURE

### Technical Problem

An aspect is to provide a Blautia massiliensis strain belonging to the Blautia sp. and being deposited under accession number KCTC 14559BP, a Blautia obeum strain belonging to the Blautia sp. and being deposited under accession number KCTC 14560BP, a Blautia wexlerae strain belonging to the Blautia sp. and being deposited under accession number KCTC 14561B, a Leuconostoc lactis strain belonging to the Leuconostoc sp. and being deposited under accession number KCTC 14580BP, or a Ruminococcus bromii strain belonging to the Ruminococcus sp. and being deposited under accession number KCTC 14579BP.

Another aspect is to provide a vesicle derived from the strain, lysate of the strain, or a culture solution of the strain.

Another aspect provides a pharmaceutical composition for preventing or treating inflammatory disease, the pharmaceutical composition including, as an active ingredient, the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing.

Another aspect is to provide a health functional food for preventing or ameliorating inflammatory disease, the health functional food including, as an active ingredient, the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing.

Another aspect is to provide a health functional food for ameliorating intestinal health, the health functional food including, as an active ingredient, the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing.

Another aspect is to provide a pharmaceutical composition for preventing or treating bacterial infection, the pharmaceutical composition including, as an active ingredient, the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing.

Another aspect is to provide a health functional food for preventing or ameliorating bacterial infection, the health functional food including, as an active ingredient, the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing.

Another aspect is to provide a cosmetic composition including the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing.

Another aspect is to provide an antibacterial composition for external preparation on skin including the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing.

Another aspect is to provide a method of preventing or treating bacterial infection, the method including administering the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing, to a subject in need of the foregoing.

Another aspect is to provide use of the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing, in preparation of a composition for preventing or treating bacterial infection.

### Technical Solution to Problem

An aspect provides a Blautia massiliensis strain belonging to the Blautia sp. and being deposited under accession number KCTC 14559BP, a Blautia obeum strain belonging to the Blautia sp. and being deposited under Accession No. KCTC 14560BP, a Blautia wexlerae strain belonging to the Blautia sp. and being deposited under accession number KCTC 14561 B, a Leuconostoc lactis strain belonging to the Leuconostoc sp. and being deposited under accession number KCTC 14580BP, or a Ruminococcus bromii strain belonging to the Ruminococcus sp. and being deposited under accession number KCTC 14579BP.

The B. massiliensis strain may be a strain including 16S rRNA of SEQ ID NO: 1. The B. obeum strain may be a strain including 16S rRNA of SEQ ID NO: 2. The B. wexlerae strain may be a strain including 16S rRNA of SEQ ID NO: 3. The L. lactis strain may be a strain containing 16S rRNA of SEQ ID NO: 4. The R. bromii strain may be a strain containing 16S rRNA of SEQ ID NO: 5.

The strains may have anti-inflammatory and/or antibacterial activity.

The strains may inhibit production of nitric oxide in inflammation-induced cells, inhibit expression of inflammatory cytokines (e.g., TNF-α or IL-6), or inhibit growth of bacteria (e.g., C. difficile). In addition, the strains may reduce inflammatory factors, such as pro-inflammatory cytokines (e.g., TNF or CCL2), induced by C. difficile, or may increase anti-inflammatory cytokines (e.g., IL-10).

Another aspect provides the B. massiliensis strain, the B. obeum strain, the B. wexlerae strain, the L. lactis strain, or the R. bromii strain, a vesicle derived from the strains, a lysis solution of the strains, a culture solution of the strains, an extract of the culture solution, or a mixture of the foregoing.

The strains are the same as described above.

The term “vesicle” as used in the present specification refers to a particle secreted from a cell and released into the extracellular space, and may include many different kinds, such as exosomes, ectosomes, microvesicles, microparticles, exosome-like vesicles, and the like. Extracellular vesicles may reflect the state of the origin cells (donor cells), exhibit various biological activities depending on which cells the extracellular vesicles are secreted from, and play an important role in intercellular interactions by transferring genetic substances and proteins between cells. In addition, cell-derived substances containing the vesicles may cause a disease or stimulate immune cells to fight against a disease, and also have an effect of helping humans break down and absorb substances that cannot be digested through metabolic processes of microorganisms. The vesicles may be, as membrane-structured vesicles, partitioned into an inside and an outside, may contain cell membrane lipids, plasma membrane proteins, nucleic acids, and cytoplasmic components, and may be smaller than original cells.

In an embodiment, the vesicles may be isolated from a cell lysate in a culture solution of the B. massiliensis strain, the B. obeum strain, the B. wexlerae strain, the L. lactis strain, or the R. bromii strain.

In an embodiment, the extracellular vesicle may have a diameter of 10 nm to 400 nm. For example, the diameter of the extracellular vesicle may be 10 nm to 400 nm, 10 nm to 350 nm, 10 nm to 300 nm, or 10 nm to 250 nm.

The term “culture solution” as used in the present specification may be used interchangeably with “culture supernatant,” “conditioned culture solution,” or “adjusted medium,” and may also refer to a whole medium containing a strain, metabolites thereof, extra nutrients, etc., obtained by culturing the strain for a certain period of time in a medium capable of supplying nutrients to enable the strain, e.g., the B. massiliensis strain, the B. obeum strain, the B. wexlerae strain, the L. lactis strain, or the R. bromii strain, to grow and survive in vitro. Also, the culture solution may refer to a culture solution obtained by removing bacterial cells from a bacterial cell culture solution obtained by culturing the strain. Meanwhile, the liquid from which bacterial cells have been removed from the culture solution is also referred to as “supernatant,” and the supernatant may be obtained by leaving the culture solution for a certain period of time and collecting only the liquid in the upper layer, except for any precipitated part in the lower layer, or by collecting the liquid at the top after removing the bacterial cells through filtration or removing a precipitate at the bottom through centrifugation. The term “bacterial cell” as used herein refers to the strain itself of the present disclosure, and may include a strain itself isolated and selected from a skin sample or the like, or a strain isolated from a culture solution obtained by culturing the strain. The bacterial cells may be obtained by centrifuging a culture solution and collecting a portion precipitated in the lower layer, or by leaving a culture solution for a certain period of time and removing the liquid at the top since the bacterial cells precipitate to the lower layer of a culture solution due to gravity.

The culture solution may contain a culture solution itself obtained by culturing the strain, a concentrate of the culture solution, or a freeze-dried product of the culture solution, or a culture supernatant obtained by removing a strain from the culture solution, a concentrate of the culture supernatant, or a freeze-dried product of the culture supernatant.

The culture solution may be obtained by culturing the strain in an appropriate medium (e.g., R2A medium or TSA medium) at any temperature above 10° C. or below 40° C. for a certain period of time, for example, 4 hours to 50 hours.

In an embodiment, the culture supernatant of the strain may be obtained by removing the strain through centrifugation or filtration of the strain culture solution.

In one or more embodiments, the concentrate may be obtained by concentrating the strain culture solution itself or the supernatant that is obtained by centrifuging of the culture solution or filtration of the culture solution by using a filter.

Culture media and culture conditions for culturing the strain may be appropriately selected or modified by those skilled in the art.

The term “lysate” as used in the present specification may refer to a product obtained by breaking the cell wall of the strain itself by using chemical or physical force.

The term “extract of culture solution” as used in the present specification refers to an extract of the culture solution or a concentrate thereof, and the extract may contain an extraction solution, a diluted or concentrated solution of the extraction solution, a dried product obtained by drying the extraction solution, or a crude or purified product of the foregoing, and a fraction obtained by fractionating the foregoing.

Another aspect provides use of the B. massiliensis strain, the B. obeum strain, the B. wexlerae strain, the L. lactis strain, or the R. bromii strain, a vesicle derived from the strains, a lysis solution of the strains, a culture solution of the strains, or a mixture of the foregoing, for amelioration, prevention, or treatment of disease.

The term “treat” as used in the present specification may refer to healing of inflammation or bacterial infection in a shorter time compared to natural healing. The treatment may include amelioration and/or alleviation of inflammation or bacterial infection. Also, the treatment may refer to healing and/or recovery of symptoms caused by inflammation or bacterial infection.

The use of the strain may include: prevention, amelioration, or treatment (anti-inflammatory activity) of inflammatory disease; prevention, amelioration, or treatment (antibacterial activity) of bacterial infection; or prevention or amelioration of intestinal health.

The inflammatory disease may include inflammation of the digestive system (gastrointestinal tract, etc.), inflammation within the eyes, inflammation within the oral cavity, inflammation of the respiratory system including the lungs, inflammation of the skin, inflammation within the cardiovascular system, inflammation of the brain, inflammation within the ears, and the like.

In particular, the inflammatory disease may be one selected from the group consisting of inflammatory bowel diseases (IBDs); irritable bowel syndrome; Behcet's disease; enteritis; Crohn's disease; ulcerative colitis; vasculitis; mucositis; stomatitis; peri-implantitis; periodontitis; pulpitis; gingivitis; pneumonia; dermatitis; atopic dermatitis; contact dermatitis; CREST syndrome; dermatitis herpetiformis; dermatomyositis; systemic scleroderma; erythema nodosum; Henoch-Schonlein purpura; Hidradenitis suppurativa; Lichen planus; Majeed syndrome; Schnitzler syndrome; psoriasis; eczema; acne; mouth ulcers; uveitis; pharyngitis; tonsillitis; otitis including tympanitis; psoriatic arthritis; synovitis; meningitis; encephalitis; Bickerstaff's encephalitis; ncephalomyelitis; spondylitis; osteomyelitis; Guillain-barre syndrome; myelitis; neuromyelitis optica; cystitis; acute inflammation on an infection or wound site; nephriti; and glomerulonephritis.

Also, the amelioration of intestinal health may include an aid to growth of beneficial intestinal bacteria and inhibition of harmful bacterial, an aid to intestinal health by immunity control, or an aid to smooth bowel movements.

The term “antibacterial agent” as used in the present specification refers to a substance that can: (i) inhibit, reduce, or prevent the growth of bacteria; (ii) inhibit or reduce the ability of bacteria to cause infection in a subject; or (iii) inhibit or reduce the ability of bacteria to grow or maintain infectiousness in an environment.

Examples of the bacterial infection may include infections caused by gram-positive bacteria or gram-negative bacteria. Specifically, the bacterial infection may include infection caused by bacteria belonging to the Clostridium (C.) sp., the Helicobacter sp., the Escherichia sp., the Salmonella sp., the Staphylococcus sp., the Streptococcus sp., the Haemophilus sp., the Klebsiella sp., the Moraxella sp., the Enterobacter sp., the Proteus sp., the Serratia sp., the Pseudomonas sp., the Acinetobacter sp., the Citrobacter sp., the Stenotrophomonas sp., the Bacteroides sp., the Prevotella sp., or the Fusobacterium sp. More specifically, the bacterial infection may include C. difficile infection (CDI) or C. difficile associated disease (CDAD), such as C. difficile associated diarrhea.

The composition may include the strain, a lysis solution thereof, a culture solution thereof, or an extract of the culture solution, in an amount of, based on the total weight of the composition, 0.00001 wt % to 80 wt %, for example, 0.00001 wt % to 60 wt %, 0.00001 wt % to 40 wt %, 0.00001 wt % to 30 wt %, 0.00001 wt % to 20 wt %, 0.00001 wt % to 10 wt %, 0.00001 wt % to 5 wt %, 0.05 wt % to 60 wt %, 0.05 wt % to 40 wt %, 0.05 wt % to 30 wt %, 0.05 wt % to 20 wt %, 0.05 wt % to 10 wt %, 0.05 wt % to 5 wt %, 0.1 wt % to 60 wt %, 0.1 wt % to 40 wt %, 0.1 wt % to 30 wt %, 0.1 wt % to 20 wt %, 0.1 wt % to 10 wt %, or 0.1 wt % to 5 wt %.

The expression “including as an active ingredient” as used herein refers that the strain disclosed in the present specification, a vesicle derived from the strains, a lysis solution of the strains, a culture solution of the strains, or an extract of the culture solution is added to the extent that the aforementioned effects can be exhibited. It also refers that various ingredients are added as side ingredients and formulated in various forms for drug delivery and stabilization.

In one or more embodiments, the composition may be a pharmaceutical composition.

The pharmaceutical composition may additionally include a pharmaceutically acceptable diluent or carrier. The diluent may include lactose, corn starch, soybean oil, microcrystalline cellulose, or mannitol, and a glydent may include magnesium stearate, talc, or a combination thereof. The carrier may include an excipient, a disintegrant, a binding agent, a glydent, or a combination thereof. The excipient may include microcrystalline cellulose, lactose, low-substituted hydroxy cellulose, or a combination thereof. The disintegrant may include carboxylmethyl cellulose calcium, sodium starch glycolate, anhydrous calcium hydrogen phosphate, or a combination thereof. The binding agent may include polyvinyl pyrrolidone, low-substituted hydroxypropyl cellulose, hydroxypropyl cellulose, or a combination thereof. The glydent may include magnesium stearate, silicon dioxide, talc, or a combination thereof.

The pharmaceutical composition may be formulated for oral or parenteral administration. The formulation for oral administration may include granule, powder, liquid, tablet, capsule, dry syrup, or a combination thereof. The formulation for parenteral administration may include injection.

The composition may be a health functional food composition.

For the health functional food composition, the strain or a culture solution thereof may be used alone or in combination with other foods or food ingredients, and may be appropriately used according to methods in the art. A mixing amount of the active ingredient may be appropriately determined depending on the purpose of use (prevention, health or therapeutic treatment). In general, when preparing food or beverage, the composition disclosed herein may be added in an amount of 15 parts by weight or less with respect to the raw materials. Types of the health functional food are not particularly limited. Among the types of the health functional food, a beverage composition may contain, as additive ingredients, various flavoring agents or natural carbohydrates as in general beverages. The native carbohydrate may include monosaccharides, such as glucose and fructose, disaccharides, such as maltose and sucrose, polysaccharides, such as dextrin and cyclodextrin, and sugar alcohols, such as xylitol, sorbitol, and erythriotol. For use as the sweetening agent, a natural sweetening agent, such as thaumatin and a stevia extract, a synthetic sweetening agent, such as saccharin, aspartame, and the like may be used. The health food composition may also contain a nutrient, a vitamin, an electrolyte, a flavoring agent, a colorant, pectic acid and a salt thereof, alginic acid and a salt thereof, organic acid, a protective colloidal thickener, a pH adjuster, a stabilizer, a preservative, glycerin, alcohol, and a carbonizing agent used in carbonated beverages, or a combination thereof. The health functional food composition may also include natural fruit juice and fruit flesh for preparing fruit juice beverage and vegetable beverage, or a combination thereof.

The composition may be a cosmetic composition.

The cosmetic composition may be, for example, a softening lotion, a nutrient lotion, a massage cream, a nutrient cream, an essence, a pack, a gel, an ampoule, or a skin-adhesive cosmetic formulation.

Ingredients included in the cosmetic composition may include, as an active ingredient, components that are commonly used in the cosmetic composition, in addition to the composition. For example, the components may include common auxiliary agents and carriers, such as a stabilizer, a solubilizer, a vitamin, a pigment, and a flavor.

Also, the composition may be a composition for external preparation on skin.

In the present specification, the external preparation on skin may be a cream, a gel, an ointment, a skin emulsifier, a skin suspension, a transdermal delivery patch, a drug-containing bandage, a lotion, or a combination thereof. For the external preparation on skin, ingredients used in ordinary external preparation on skin including cosmetics or pharmaceuticals, such as water-based ingredients, oil-based ingredients, powdered ingredients, alcohols, moisturizers, thickeners, ultraviolet ray absorbents, whitening agents, preservatives, antioxidants, surfactants, flavoring agents, colorants, various skin nutrients, or a combination thereof, may be appropriately blended as needed. For the external preparation on skin, a sequestrant, such as disodium edetate, trisodium edetate, trisodium citrate, sodium polyphosphate, sodium metaphosphate, gluconic acid, or the like, caffeine, tannin, verapamil, a licorice extract, glabridin, a hot water extract of caline fruit, various herbal medicines, tocopherol acetate, glycyrrhizic acid, tranexamic acid, a derivative thereof, or a medicine of a salt or the like thereof, vitamin C, magnesium ascorbyl phosphate, ascorbyl glucoside, albutin, kojic acid, or sugar, such as glucose, fluctose, trehalose, or the like, may be also appropriately blended.

Also, another aspect provides a method of preventing, ameliorating, or treating a condition of a subject, the method including treating or administering an effective amount of the composition to a subject in need thereof.

The condition of a subject may be a condition related to inflammation or a condition related to bacterial infection.

The administration may be performed by a method known in the art. The administration may be performed directly to a subject by any means, for example, intravenous, intramuscular, oral, transdermal, mucosal, intranasal, intratracheal, or subcutaneous routes. The administration may be administered systemically or locally.

The subject may be a mammal, for example, a human, a cow, a horse, a pig, a dog, a sheep, a goat, or a cat. The subject may be an individual in need of an improvement effect in a condition related to inflammation or a condition related to bacterial infection.

For the administration, the composition according to an embodiment may be administered in an amount of 0.00001 mg to 1,000 mg per subject per day, for example, 0.00001 mg to 500 mg per subject per day, 0.00001 mg to 100 mg per subject per day, 0.00001 mg to 50 mg per subject per day, 0.00001 mg to 25 mg per subject per day, 1 mg to 1,000 mg per subject per day, 1 mg to 500 mg per subject per day, 1 mg to 100 mg per subject per day, 1 mg to 50 mg per subject per day, 1 mg to 25 mg per subject per day, 5 mg to 1,000 mg per subject per day, 5 mg to 500 mg per subject per day, 5 mg to 100 mg per subject per day, 5 mg to 50 mg per subject per day, 5 mg to 25 mg per subject per day, 10 mg to 1,000 mg per subject per day, 10 mg to 500 mg per subject per day, 10 mg to 100 mg per subject per day, 10 mg to 50 mg per subject per day, or 10 mg to 25 mg per subject per day. However, the dosage may be variously prescribed depending on factors, such as a formulation method, an administration method, age, weight, gender, and medical conditions of a patient, food, administration time, an administration route, an excretion rate, and response sensitivity, and a person skilled in the art may appropriate adjust the dosage in consideration of these factors. The number of administration may be once a day or at least twice a day within the range of clinically acceptable side effects, and the administration may be performed at a single site or at least two sites, daily or every 2 days to 5 days. The total number of administration days may be 1 day to 30 days per treatment. As needed, the same treatment may be repeated after the a suitable period of time. For animals other than humans, the same dosage per kg as for humans may be used, or for example, a dosage converted from the aforementioned dosage by the volume ratio (e.g., average value) of the organ (e.g., heart, etc.) between a target animal and the human may be used.

### Advantageous Effects of Disclosure

According to an aspect, the novel strain and the vesicle derived therefrom have effects that can be useful in the prevention, amelioration, or treatment of a condition related to inflammation or a condition related to bacterial infection.

### BRIEF DESCRIPTION OF DRAWINGS

FIG. 1 is a graph showing the amount of nitric oxide produced according to cellular treatment on vesicles of a strain according to an embodiment, N: negative control, P: untreated control, EV: vesicles of Example 2.

FIG. 2 is a graph showing the protein expression levels of pro-inflammatory cytokines (TNF and IL-6) and an anti-inflammatory cytokine (IL-10) according to the cellular treatment with vesicles of a strain according to an embodiment; N: negative control, P: untreated control, EV: vesicles of Example 2.

FIG. 3 is a graph showing the culture rate of C. difficile in a culture solution of a strain according to an embodiment and a culture solution of Blautia (B. ) massiliensis reference strain.

FIG. 4 is a graph showing the culture rate of C. difficile in vesicles of a strain according to an embodiment.

FIG. 5 is a graph showing the cytotoxicity results of vesicles of a strain according to an embodiment; N: negative control, P: untreated control, EV: vesicles of Example 2.

FIG. 6 is a graph showing the cytotoxicity results of vesicles of a strain according to an embodiment; CdEV: vesicles of C. difficile, BBH020: vesicles of the strain of Example 1, Type strain: vesicles of B. massiliensis reference strain.

FIG. 7 is a graph showing the activity of vesicles of a strain according to an embodiment to reduce inflammation induced by C. difficile; CdEV: vesicles of C. difficile, BBH020: vesicles of the strain of Example 1, Type strain: vesicles of B. massiliensis reference strain.

FIG. 8 is a graph showing the amount of nitric oxide produced according to cellular treatment on vesicles of a strain according to an embodiment; N: negative control, P: untreated control, EV: vesicles of Example 2.

FIG. 9 is a graph showing the protein expression levels of pro-inflammatory cytokines (TNF and IL-6) and an anti-inflammatory cytokine (IL-10) according to the cellular treatment with vesicles of a strain according to an embodiment; N: negative control, P: untreated control, EV: vesicles of Example 2.

FIG. 10 is a graph showing the culture rate of C. difficile in a culture solution of a strain according to an embodiment.

FIG. 11 is a graph showing the culture rate of C. difficile in vesicles of a strain according to an embodiment.

FIG. 12 is a graph showing the cytotoxicity results of vesicles of a strain according to an embodiment; N: negative control, P: untreated control, EV: vesicles of Example 2.

FIG. 13 is a graph showing the cytotoxicity results of vesicles of a strain according to an embodiment; CdEV: vesicles of C. difficile, BBH021: vesicles of the strain of Example 1, Type strain: vesicles of B. obeum reference strain.

FIG. 14 is a graph showing the activity of vesicles of a strain according to an embodiment to reduce inflammation induced by C. difficile; CdEV: vesicles of C. difficile, BBH021: vesicles of the strain of Example 1, Type strain: vesicles of B. obeum reference strain.

FIG. 15 is a graph showing the amount of nitric oxide produced according to cellular treatment on vesicles of a strain according to an embodiment; N: negative control, P: untreated control, EV: vesicles of Example 2.

FIG. 16 is a graph showing the protein expression levels of pro-inflammatory cytokines (TNF and IL-6) and an anti-inflammatory cytokine (IL-10) according to the cellular treatment with vesicles of a strain according to an embodiment; N: negative control, P: untreated control, EV: vesicles of Example 2.

FIG. 17 is a graph showing the culture rate of C. difficile in vesicles of a strain according to an embodiment and vesicles of B. wexlerae reference strain.

FIG. 18 is a graph showing the cytotoxicity results of vesicles of a strain according to an embodiment; N: negative control, P: untreated control, EV: vesicles of Example 2.

FIG. 19 is a graph showing the cytotoxicity results of vesicles of a strain according to an embodiment; CdEV: vesicles of C. difficile, BBH022: vesicles of the strain of Example 1, Type strain: vesicles of B. wexlerae reference strain.

FIG. 20 is a graph showing the activity of vesicles of a strain according to an embodiment to reduce inflammation induced by C. difficile; CdEV: vesicles of C. difficile, BBH022: vesicles of the strain of Example 1, Type strain: vesicles of B. wexlerae reference strain.

FIG. 21 is a graph showing the amount of nitric oxide produced according to cellular treatment on vesicles of a strain according to an embodiment; N: negative control, P: untreated control, EV: vesicles of Example 2.

FIG. 22 is a graph showing the protein expression levels of pro-inflammatory cytokines (IL-6 and CCL2) and an anti-inflammatory cytokine (IL-10) according to the cellular treatment with vesicles of a strain according to an embodiment; N: negative control, P: untreated control, EV: vesicles of Example 2.

FIG. 23 is a graph showing the culture rate of C. difficile in a culture solution of a strain according to an embodiment.

FIG. 24 is a graph showing the culture rate of C. difficile in vesicles of a strain according to an embodiment and vesicles of Leuconostoc lactis reference strain.

FIG. 25 is a graph showing the cytotoxicity results of vesicles of a strain according to an embodiment; N: negative control, P: untreated control, EV: vesicles of Example 2.

FIG. 26 is a graph showing the cytotoxicity results of vesicles of a strain according to an embodiment; CdEV: vesicles of C. difficile, BBH018: vesicles of the strain of Example 1, Type strain: vesicles of L. lactis reference strain.

FIG. 27 is a graph showing the activity of vesicles of a strain according to an embodiment to reduce inflammation induced by C. difficile; CdEV: vesicles of C. difficile, BBH018: vesicles of the strain of Example 1, Type strain: vesicles of L. lactis reference strain.

FIG. 28 is a graph showing the protein expression levels of pro-inflammatory cytokines (TNF-α) and an anti-inflammatory cytokine (IL-10) according to the cellular treatment with vesicles of a strain according to an embodiment; N: negative control, P: untreated control, EV: vesicles of Example 2.

FIG. 29 is a graph showing the culture rate of C. difficile in the supernatant of a strain according to an embodiment and the supernatant of Ruminococcus bromii reference strain (R. bromii ATCC27255).

FIG. 30 is a graph showing the cytotoxicity results of vesicles of a strain according to an embodiment; N: negative control, P: untreated control, EV: vesicles of Example 2.

FIG. 31 is a graph showing the cytotoxicity results of a strain according to an embodiment or a reference strain after performing treatment with vesicles of C. difficile; CdEV: vesicles of C. difficile, BBH015: vesicles of the strain of Example 1, Type strain: vesicles of R. bromii reference strain.

FIG. 32 is a graph showing the activity of vesicles of a strain according to an embodiment to reduce inflammation induced by C. difficile; CdEV: vesicles of C. difficile, BBH015: vesicles of the strain of Example 1, Type strain: vesicles of R. bromii reference strain.

### MODE OF DISCLOSURE

Hereinafter, the present disclosure will be described in more detail with reference to Examples below. However, these Examples are for illustrative purposes of one or more embodiments, and the scope of the present disclosure is not limited thereto.

### Example 1. Isolation and Identification of Strains

To isolate and identify strains from feces of healthy people, the following procedures were performed.

First, 1 g of feces collected from a healthy person was mixed with 10 ml of 1× phosphate buffer saline (PBS), and then vortexed to suspend the feces.

Next, to remove undigested food and small particle substances, the fecal suspension was filtered through a cell strainer. The filtered fecal suspension was serially diluted and spread at a concentration of 10⁻⁶ to 10⁻⁸ on a brain heart infusion-supplemented (BHIs) 20% rumen fluid medium plate, and then, bacteria were selected after culturing the same at 37° C. for 3 days or more. Alternatively, the filtered fecal suspension was serially diluted and spread at a concentration of 10⁻⁶ to 10⁻⁸ on a fastidious anaerobe broth (FAB)-supplemented 5% sheep blood medium plate, and then, bacteria were selected after culturing the same at 37° C. for 3 days or more. Alternatively, the filtered fecal suspension was serially diluted and spread at a concentration of 10⁻⁶ to 10⁻⁸ on a BHIs 5% sheep blood medium plate, and then, bacteria were selected after culturing the same at 37° C. for 3 days or more. Alternatively, the filtered fecal suspension was serially diluted and spread at a concentration of 10-4 to 10⁻⁶ on a deMan-Rogosa-Sharpe-supplemented vancomycin medium plate, and then, bacteria were selected after culturing the same at 37° C. for 3 days or more. Alternatively, the filtered fecal suspension was serially diluted and spread at a concentration of 10⁻⁶ to 10⁻⁸ on a YCFA (DSMZ 1611) medium plate, and then, the bacteria were selected after culturing the same at 37° C. for 3 days or more. PCR amplification was performed on colonies for which the culture was completed the cultured colonies, as well as on colonies for which isolation culture was completed. Then, the nucleic sequences of the 16S rRNA regions determined among the colonies cultured for isolation were compared with other strains registered with a BLAST program available on the EzBioCloud Database website (ChunLab, Ez Taxon).

As a result of the comparative analysis, the B. massiliensis BBH 020 strain with 97% homology was isolated. The selected B. massiliensis BBH 020 strain was deposited at the Korean Collection for Type Cultures (KCTC) on May 3, 2021 and given an accession number of KCTC14559BP. The B. massiliensis BBH 020 strain has the 16S rRNA sequence of SEQ ID NO: 1 (complementary DNA).

As a result of the comparative analysis, the B. obeum BBH 021 strain with 98% homology was isolated. The selected B. obeum BBH 021 strain was deposited at the KCTC on May 3, 2021 and given an accession number of KCTC14560BP. The B. obeum BBH 021 strain has the 16S rRNA sequence of SEQ ID NO: 2 (complementary DNA).

As a result of comparative analysis, the B. wexlerae BBH 022 strain with 99% homology was isolated. The selected B. wexlerae BBH 022 strain was deposited at the KCTC on May 3, 2021 and was given an accession number of KCTC14561BP. The B. wexlerae BBH 022 strain has a 16S rRNA sequence of SEQ ID NO: 3 (complementary DNA).

As a result of comparative analysis, the L. lactis BBH 018 strain with 99% homology was isolated. The selected L. lactis BBH 018 strain was deposited at the KCTC on May 25, 2021 and given an accession number of KCTC14580BP. The L. lactis BBH 018 strain has the 16S rRNA sequence of SEQ ID NO: 4 (complementary DNA).

As a result of comparative analysis, the R. bromii BBH 015 strain with 98% homology was isolated. The selected R. bromii BBH 015 strain was deposited at the KCTC on May 25, 2021 and given an accession number of KCTC14579BP. The R. bromii BBH 015 strain has the 16S rRNA sequence of SEQ ID NO: 5 (complementary DNA).

### Example 2. Isolation of Vesicles

Vesicles of the strain isolated in Example above were isolated.

In detail, to prepare vesicles, the isolated strain was cultured for 2 days in a PYG broth (DSMZ 104) at 37° C. under anaerobic conditions. Afterwards, the culture solution was centrifuged at 5,000×g for 20 minutes to remove bacterial debris. Afterwards, the resulting culture solution was filtered first through a 0.45 um filter, and filtered again through a 0.22 um filter. Then, substances over 30 kda were concentrated by using a ultrafiltration (UF) system (CNS) utilizing a filter (Sartorius, Cassete Sartocon Slice Hydrosart filter), and vesicles of the isolated strain were isolated therefrom.

### Experimental Example 1. Anti-Inflammatory Activity Assay

The anti-inflammatory activity of the isolated vesicles of the strain in Example 2 was analyzed.

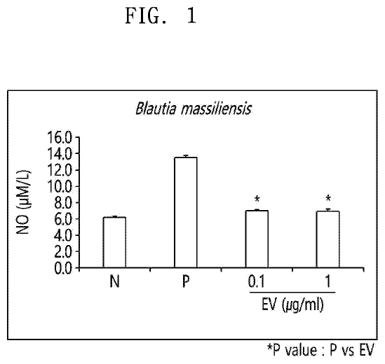

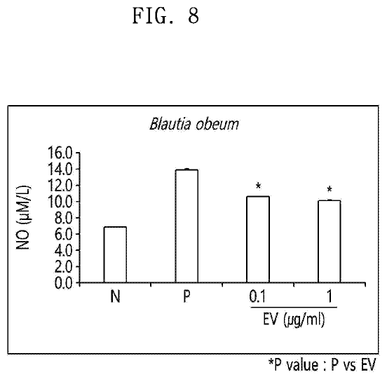

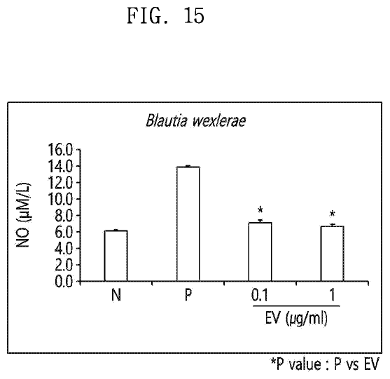

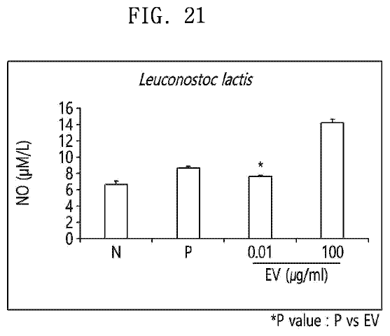

First, to evaluate the anti-inflammatory activity, a reduction in nitric oxide (NO) production was measured. Mouse macrophage Raw264.7 cells were cultured in a Dulbecco Modified Eagle Medium (DMEM) supplemented with 10% fetal bovine serum (FBS) and 1% antibiotics (100 U/mL penicillin and 100 μg/mL streptomycin), in the presence of 5% CO₂ at 37° C. Afterwards, 300 μL of the Raw 264.7 cells were dispensed into a 48-well plate at a concentration of 5×104 cells/well, and then cultured in a CO₂ incubator at 37° C. for 24 hours. The supernatant in each well was discarded, and a medium supplemented with 10 μg/ml of lipopolysaccharide (LPS) was added thereto to induce inflammation, and the cells were cultured additionally for 4 hours. Next, the supernatant containing the LPS was discarded, and the vesicles were treated by addition to the medium at a concentration of 0.01 μg/ml, 0.1 μg/ml, 1 μg/ml, or 100 μg/ml, and then, the cells were cultured at 37° C. for 16 hours. Afterwards, 50 μL of the supernatant in the well was mixed with 50 μL of Griess reagent, and the mixture was allowed for a reaction at room temperature for 10 minutes. Then, the amount of NO produced was measured by measuring absorbance at 540 nm by using a plate reader, and the results are shown in FIGS. 1, 8, 15, and 21 for each strain.

FIGS. 1, 8, 15, and 21 are each a graph showing the amount of NO produced according to cellular treatment on the vesicle of the strain according to an embodiment; N: negative control, P: untreated control, EV: vesicles of Example 2.

As shown in the figures, it was found that the vesicles of the strain according to an embodiment significantly reduced the amount of NO produced in the inflammation-induced cells.

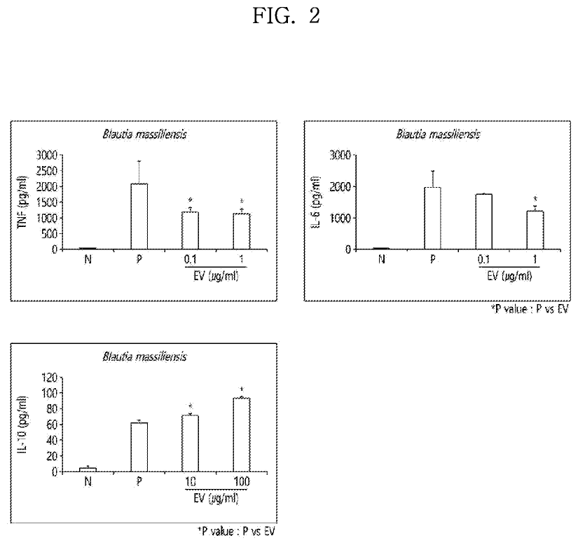

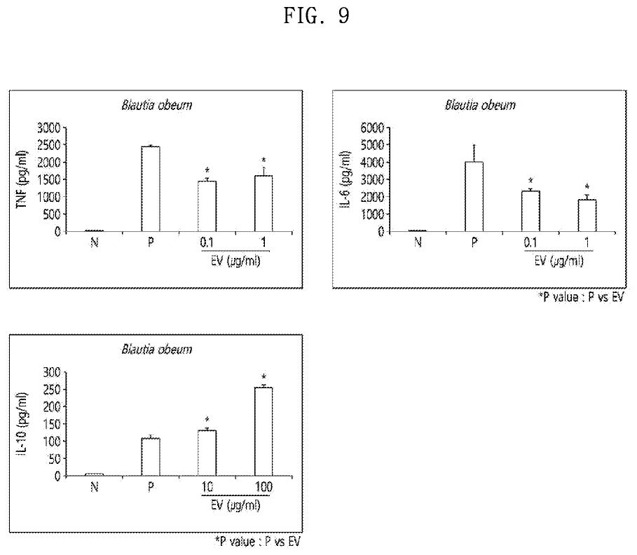

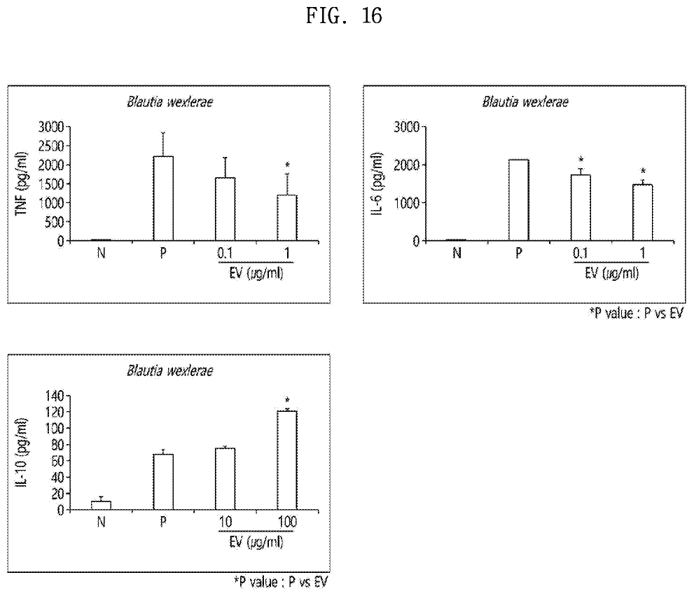

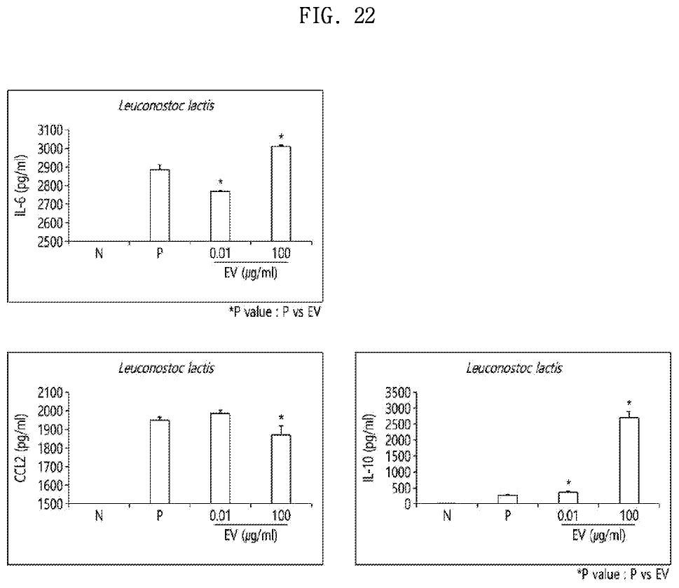

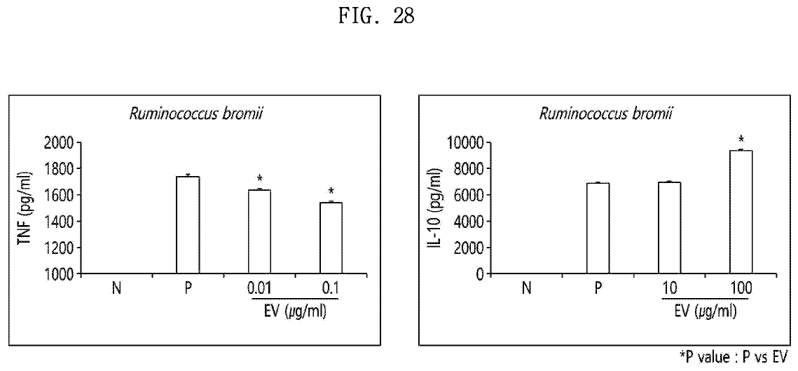

Next, the activity of the vesicles of the strain on the suppression of pro-inflammatory cytokines and on the promotion of anti-inflammatory cytokines was measured. In detail, the Raw264.7 cells treated with LPS in the same manner as described above were treated with the vesicles at a concentration of 0.01 μg/ml, 0.1 μg/ml, 1 μg/ml, or 100 μg/ml, and then cultured at 37° C. for 16 hours. Afterwards, the protein expression levels of the pro-inflammatory cytokines, TNF, IL-6, and CCL2, in the cells were measured by measuring absorbance at 450 nm by using an ELISA kit (BD bioscience, USA) according to the instructions of the manufacturer. In addition, after the vesicles were treated at a concentration of 10 μg/ml or 100 μg/ml in the same manner as described above, the protein expression level of the anti-inflammatory cytokine, IL-10, was measured, and the results are shown in FIGS. 2, 9, 16, 22, and 28 for each strain.

FIGS. 2, 9, 16, 22, and 28 are each a graph showing the protein expression levels of the pro-inflammatory cytokines and the anti-inflammatory cytokine according to the cellular treatment with the vesicles of the strain according to an embodiment; N: negative control, P: untreated control, EV: vesicles of Example 2.

As shown in the figures, it was confirmed that the vesicle of the strain according to an embodiment significantly reduced the pro-inflammatory cytokines compared to the untreated control group and significantly increased the anti-inflammatory cytokine compared to the untreated control group.

The results above refer that the strains according to an embodiment may be useful in the prevention, amelioration, or treatment of inflammatory diseases, especially inflammatory bowel disease or irritable bowel syndrome.

### Experimental Example 2. Antibacterial Activity Assay

The antibacterial activity of the isolated vesicles of the strain in Example 2 was analyzed.

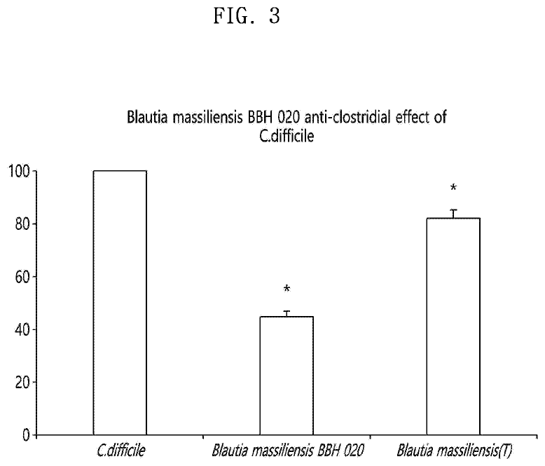

In detail, to analyze the antibacterial activity of the strains of Example 1 on C. difficile, each of the strains of Example 1 and C. difficile were pre-cultured in 5 ml of each of a PYG broth and an RCM broth, and adjusted to an OD (600 nm) of 0.5 by using a spectrophotometer. Afterwards, the culture solution of each of the strains was inoculated at a 1% ratio into 30 ml of each of a PYG broth and an RCM broth, and the strains were each cultured at 37° C. for 48 hours under anaerobic conditions. The cultured strain was centrifuged at 4,000 RPM for 10 minutes to separate the pellets from the supernatant. The pellets were then washed three times with PBS, re-suspended, and adjusted to an OD of 0.5. After C. difficile and each of the strains of Example 1 were inoculated at the same ratio into 30 ml of the RCM broth, the culture rate of the C. difficile was measured according to the colony forming unit calculation, and the results are shown in FIGS. 3. 10, 23, and 29 for each strain.

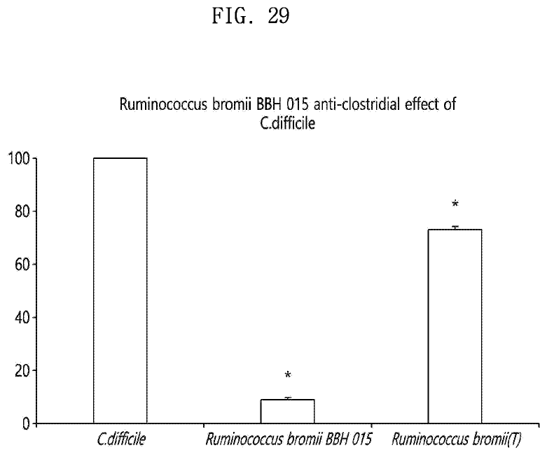

In addition, the antibacterial activity of the B. massiliensis reference strain (B. massiliensis DSM101187) and the R. bromii reference strain (R. bromii ATCC27255) on C. difficile was analyzed in the same manner as described above, and the results thereof are shown in FIGS. 3 and 29 for each strain.

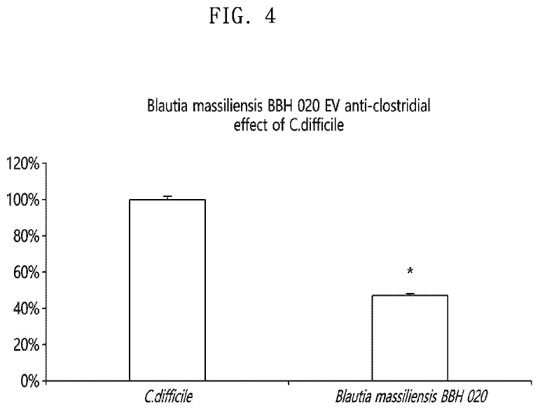

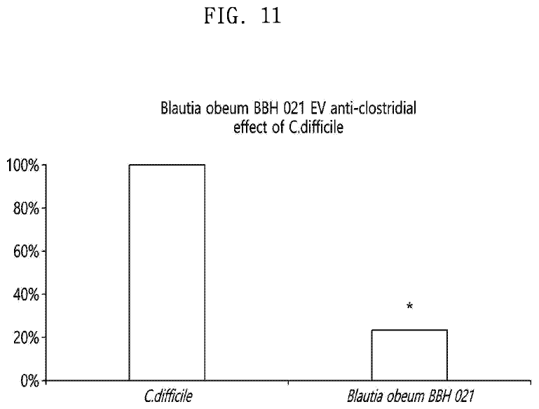

To analyze the antibacterial activity of the vesicles of the strains of Example above on C. difficile, the culture supernatant of each of the strain was centrifuged at 5,000×g for 20 minutes to isolate the vesicles. The separated supernatant was then concentrated by using a centrifuge tube (Amicon Ultra-15 Centrifuge Filter Unit). After C. difficile was cultured in a PYG broth for 48 hours and adjusted to an OD of 0.5, the C. difficile was inoculated at a ratio of 10% into the vesicles of each of the strains of Examples above, and the strain was cultured under anaerobic conditions for 1 day. Afterwards, the culture rate of the C. difficile was measured spectrophotometrically, and the results thereof are shown in FIGS. 4, 11, 17, and 24 for each strain.

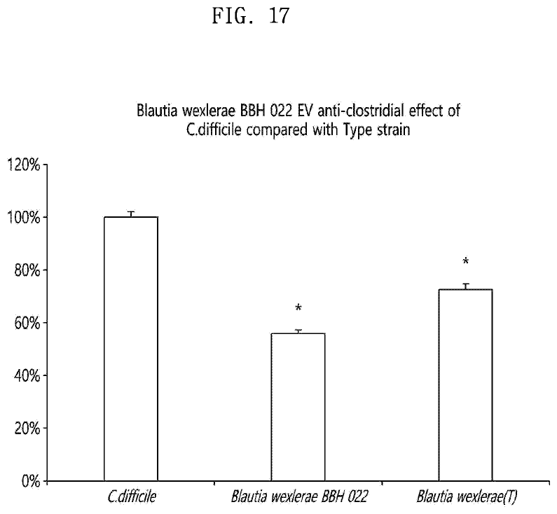

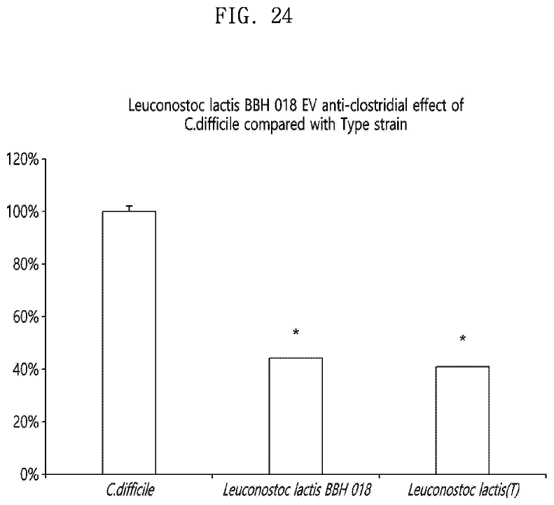

In addition, the antibacterial activity of the vesicles of the B. wexlerae reference strain (B. wexlerae DSM119850) and the L. lactis reference strain (L. lactis ATCC19256) on C. difficile was analyzed in the same manner as described above, and the results thereof are shown in FIGS. 17 and 24 for each strain.

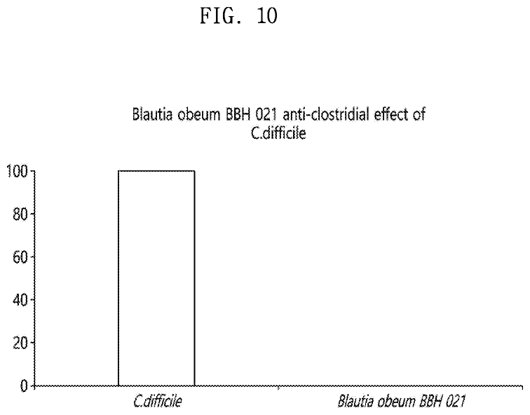

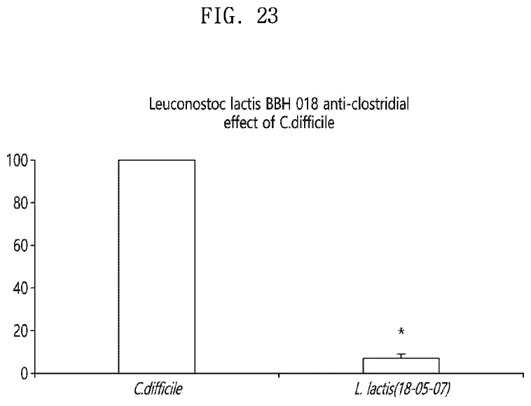

FIGS. 3, 10, 23, and 29 are each a graph showing the culture rate of C. difficile in the culture solution of each of the strains according to an embodiment.

FIGS. 4, 11, 17, and 24 are each a graph showing the culture rate of C. difficile in the vesicles of each of the strains according to an embodiment.

As shown in FIGS. 3, 10, 23, and 29, it was confirmed that the culture solution of each of the strains according to an embodiment significantly reduced the culture rate of the C. difficile.

As shown in FIGS. 3 and 29, it was confirmed that the culture solution of each of the strains according to an embodiment significantly reduced the culture rate of the C. difficile, compared to the reference strain.

As shown in FIGS. 4, 11, 17, and 24, it was confirmed that the vesicles of each of the strains according to an embodiment significantly reduced the culture rate of the C. difficile.

As shown in FIGS. 17 and 24, it was confirmed that the vesicles of each of the strains according to an embodiment significantly reduced the culture rate of the C. difficile, compared to the reference strain.

The results above refer that the strains according to an embodiment and/or the vesicles derived therefrom have the antibacterial activity against bacteria, for example, gram-negative bacteria. In particular, the results above refer that the strains according to an embodiment and/or the vesicles derived therefrom may be useful in the prevention, amelioration, or treatment of C. difficile infection (CDI), or irritable bowel syndrome caused by CDI.

### Experimental Example 3. Cytotoxicity Test

A cell counting kit-8 (CCK-8, Abbkine, China) was used to test the cytotoxicity of the isolated vesicles of the strain in Example 2.

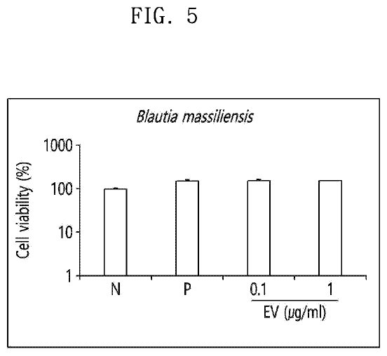

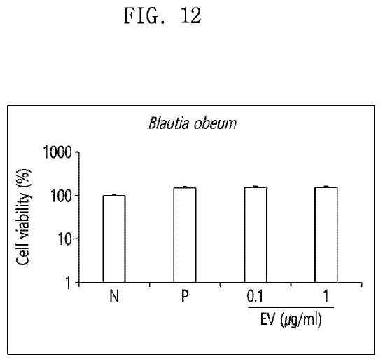

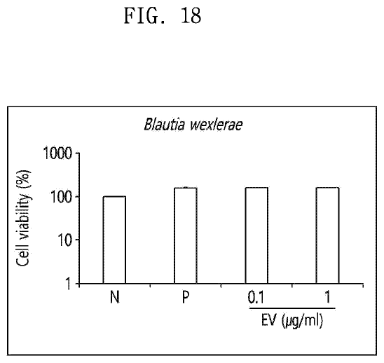

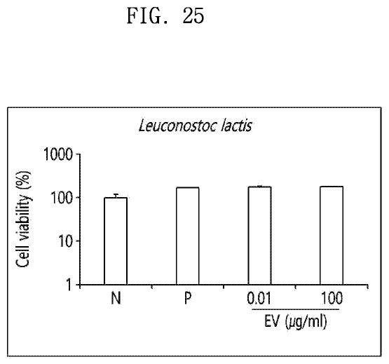

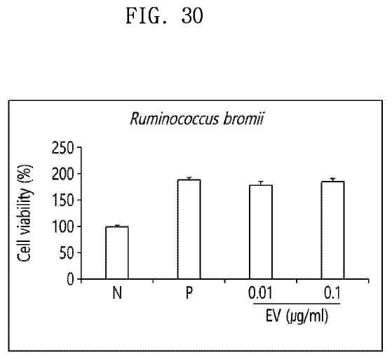

In detail, 300 μL of the Raw 264.7 cells were dispensed into a 48-well plate at a concentration of 5×10⁴ cells/well, and then cultured in a CO₂ incubator at 37° C. for 24 hours. The supernatant in each well was discarded, and a medium supplemented with 10 μg/ml of LPS was added thereto to induce inflammation, and the cells were cultured additionally for 4 hours. Next, the supernatant containing the LPS was discarded, and the vesicles were treated by addition to the medium at a concentration of 0.01 μg/ml, 0.1 μg/ml, 1 μg/ml, or 100 μg/ml, and then, the cells were cultured at 37° C. for 16 hours. Afterwards, the supernatant was removed, and 300 μl of a medium containing a 10% CCK-8 solution was added to each well, and the cells were allowed for a reaction for 4 hours. After 4 hours, the cell viability was measured by measuring the concentration of water-soluble formazan produced by succinate dehydrogenase in the mitochondria of living cells by measuring the absorbance at 450 nm, and the results thereof are shown in FIGS. 5, 12, 18, 25, and 30 for each strain.

FIGS. 5, 12, 18, 25, and 30 are each a graph showing the cytotoxicity results of the vesicles of the strain according to an embodiment; N: negative control, P: untreated control, EV: vesicles of Example 2.

As shown in the figures, it was found that the vesicles of the strain according to an embodiment significantly showed no cytotoxicity at the concentrations above.

### Experimental Example 4. Analysis of Activity to Reduce Inflammation Induced by C. difficile

The effects of the vesicles of the strain isolated in Example 1 in the reduction of the pro-inflammatory cytokines and the increase of the anti-inflammatory cytokine, induced by C. difficile were confirmed.

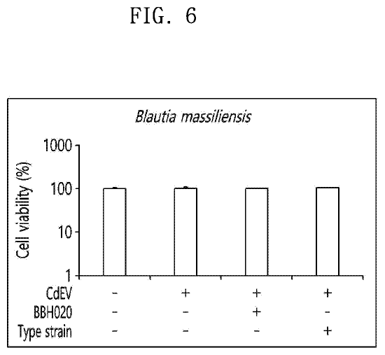

First, mouse macrophage Raw264.7 cells were treated with 10 ng/ml of vesicles of C. difficile (CdEV) in the same manner as in Experimental Example 1, and then cultured at 37° C. for 4 hours. Afterwards, the B. massiliensis reference strain (DSM101187), the B. obeum reference strain (DSM25238), the B. wexlerae reference strain (DSM19850), the L. lactis reference strain (ATCC19256), and the R. bromii reference strain (ATCC27255) were each treated with the vesicles of each of the strains of Example 1 at a concentration of 0.01 μg/ml, 0.1 μg/ml, 1 μg/ml, or 100 μg/ml, and then cultured at 37° C. for 16 hours. Next, the cell viability was measured by using the same method as in Experimental Example 2, and the results thereof are shown in FIGS. 6, 13, 19, 26, and 31 for each strain.

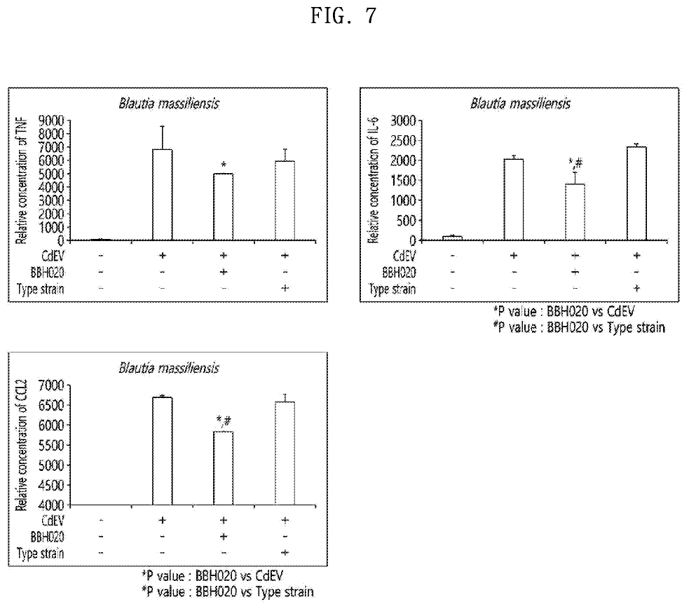

In addition, the relative concentrations of the cytokines, TNF, IL-6, CCL2, and IL-10, in the cells were measured by measuring the absorbance at 450 nm by using an ELISA kit (BD bioscience, USA) according to the instructions of the manufacturer, and the results are shown in FIGS. 7, 14, 20, 27, and 32 for each strain.

FIG. 6 is a graph showing the cytotoxicity results of the vesicles of the strain according to an embodiment; CdEV: vesicles of C. difficile, BBH020: vesicles of the strain of Example 1, Type strain: vesicles of B. massiliensis reference strain.

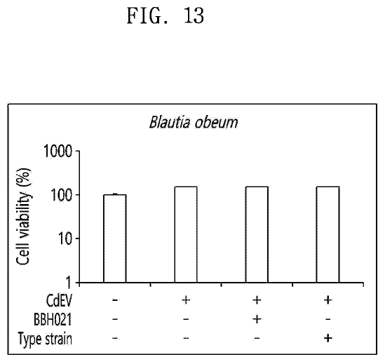

FIG. 13 is a graph showing the cytotoxicity results of the vesicles of the strain according to an embodiment; CdEV: vesicles of C. difficile, BBH021: vesicles of the strain of Example 1, Type strain: vesicles of B. obeum reference strain.

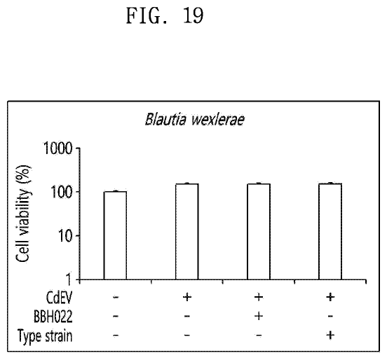

FIG. 19 is a graph showing the cytotoxicity results of the vesicles of the strain according to an embodiment; CdEV: vesicles of C. difficile, BBH022: vesicles of the strain of Example 1, Type strain: vesicles of B. wexlerae reference strain.

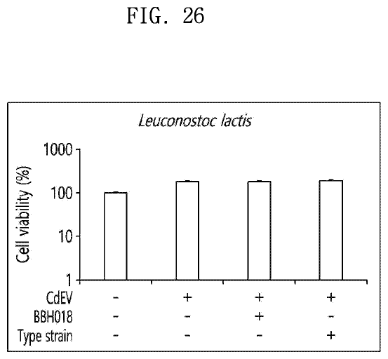

FIG. 26 is a graph showing the cytotoxicity results of the vesicles of the strain according to an embodiment; CdEV: vesicles of C. difficile, BBH018: vesicles of the strain of Example 1, Type strain: vesicles of L. lactis reference strain.

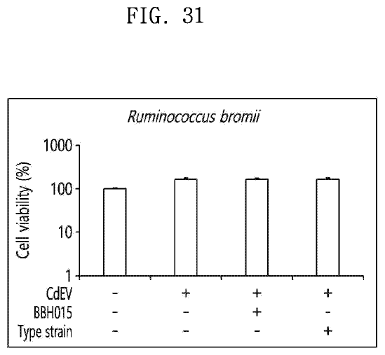

FIG. 31 is a graph showing the cytotoxicity results of the vesicles of the strain according to an embodiment; CdEV: vesicles of C. difficile, BBH015: vesicles of the strain of Example 1, Type strain: vesicles of R. bromii reference strain.

FIG. 7 is a graph showing the activity of the vesicles of the strain according to an embodiment to reduce inflammation induced by C. difficile; CdEV: vesicles of C. difficile, BBH020: vesicles of the strain of Example 1, Type strain: vesicles of B. massiliensis reference strain.

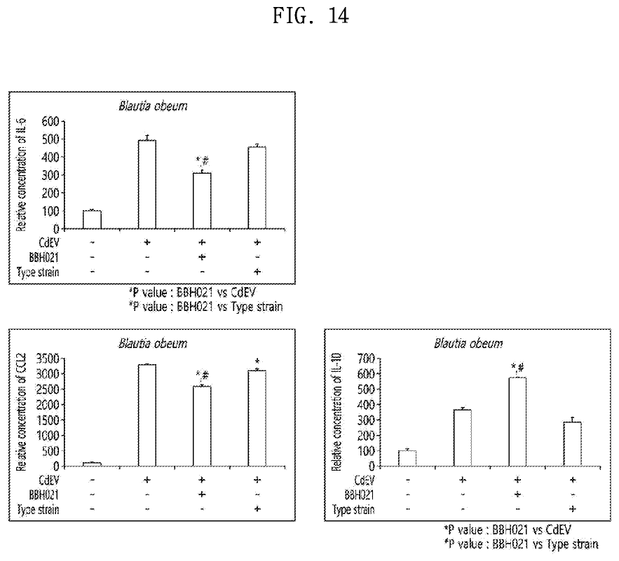

FIG. 14 is a graph showing the activity of the vesicles of the strain according to an embodiment to reduce inflammation induced by C. difficile; CdEV: vesicles of C. difficile, BBH021: vesicles of the strain of Example 1, Type strain: vesicles of B. obeum reference strain.

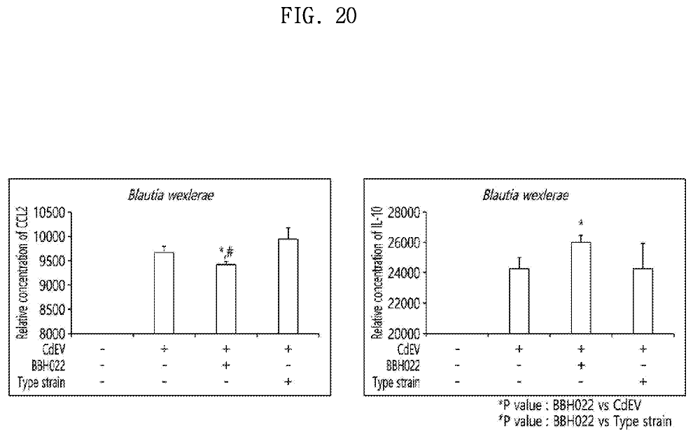

FIG. 20 is a graph showing the activity of the vesicles of the strain according to an embodiment to reduce inflammation induced by C. difficile; CdEV: vesicles of C. difficile, BBH022: vesicles of the strain of Example 1, Type strain: vesicles of B. wexlerae reference strain.

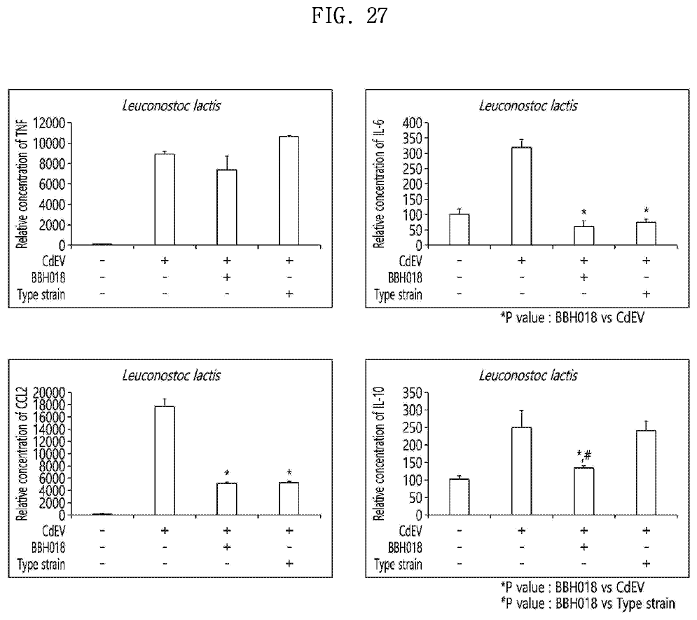

FIG. 27 is a graph showing the activity of the vesicles of the strain according to an embodiment to reduce inflammation induced by C. difficile; CdEV: vesicles of C. difficile, BBH018: vesicles of the strain of Example 1, Type strain: vesicles of L. lactis reference strain.

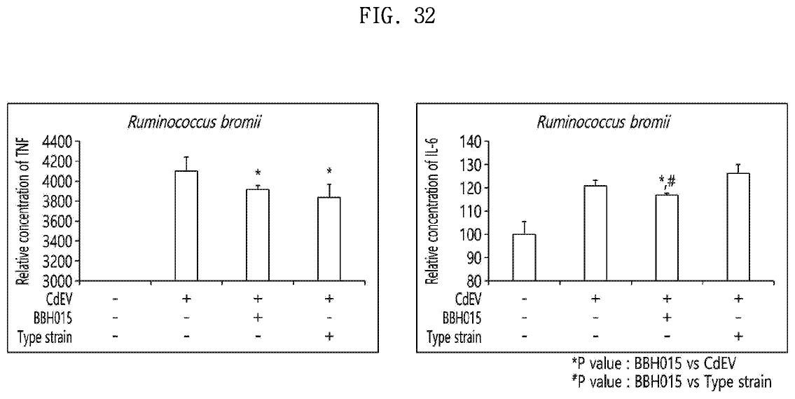

FIG. 32 is a graph showing the activity of the vesicles of the strain according to an embodiment to reduce inflammation induced by C. difficile; CdEV: vesicles of C. difficile, BBH015: vesicles of the strain of Example 1, Type strain: vesicles of R. bromii reference strain.

As shown in FIGS. 6, 13, 19, 26, and 31, it was confirmed that all the strains according to an embodiment and the reference strains showed no cytotoxicity.

Also, as shown in FIGS. 7, 14, 20, 27, and 32, it was confirmed that the vesicles of each of the strain according to an embodiment significantly reduced the amounts of the pro-inflammatory cytokines, TNF, IL-6, and CCL2, that have increased by the vesicles of C. difficile compared to the reference strains, and significantly increased the amount of the anti-inflammatory cytokine, IL-10, compared to the reference strains.

The results above refer that the strains according to an embodiment and/or the vesicles derived therefrom had significant anti-inflammatory activity, and thus may be useful in the prevention, amelioration, or treatment of CDI or irritable bowel syndrome caused by CDI.

### [Accession Number]

Name of depositary institution: Korean Collection for Type Cultures (overseas)

Accession number: KCTC 14559BP

Accession date: 20210503

_

### [Accession Number]

Name of depositary institution: Korean Collection for Type Cultures (overseas)

Accession number: KCTC 14560BP

Accession date: 20210503

_

### [Accession Number]

Name of depositary institution: Korean Collection for Type Cultures (overseas)

Accession number: KCTC 14561BP

Accession date: 20210503

_

### [Accession Number]

Name of depositary institution: Korean Collection for Type Cultures (overseas)

_

### [Accession Number]

Name of depositary institution: Korean Collection for Type Cultures (overseas)

Accession number: KCTC 14579BP

Accession date: 20210525

_

1. A Blautia massiliensis strain belonging to the Blautia sp. and being deposited under accession number KCTC 14559BP, a Blautia obeum strain belonging to the Blautia sp. and being deposited under accession number KCTC 14560BP, a Blautia wexlerae strain belonging to the Blautia sp. and being deposited under accession number KCTC 14561 B, a Leuconostoc lactis strain belonging to the Leuconostoc sp. and being deposited under accession number KCTC 14580BP, or a Ruminococcus bromii strain belonging to the Ruminococcus sp. and being deposited under accession number KCTC 14579BP.

2. The strain of claim 1, wherein the strain has antibacterial or anti-inflammatory activity.

3. The strain of claim 1, wherein the strain comprises 16s rRNA of SEQ ID NO: 1, SEQ ID NO: 2, SEQ ID NO: 3, SEQ ID NO: 4, or SEQ ID NO: 5.

4. A vesicle derived from the strain of claim 1, wherein a lysis solution of the strain of claim 1, or a culture solution of the strain of claim 1.

5. A pharmaceutical composition for preventing or treating inflammatory disease, the pharmaceutical composition comprising, as an active ingredient, a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing.

6. The pharmaceutical composition of claim 5, wherein the inflammatory disease is one selected from the group consisting of inflammatory bowel diseases (IBDs); irritable bowel syndrome; Behcet's disease; enteritis; Crohn's disease; ulcerative colitis; vasculitis; mucositis; stomatitis; peri-implantitis; periodontitis; pulpitis; gingivitis; pneumonia; dermatitis; atopic dermatitis; contact dermatitis; CREST syndrome; dermatitis herpetiformis; dermatomyositis; systemic scleroderma; erythema nodosum; Henoch-Schonlein purpura; Hidradenitis suppurativa; Lichen planus; Majeed syndrome; Schnitzler syndrome; psoriasis; eczema; acne; mouth ulcers; uveitis; pharyngitis; tonsillitis; otitis including tympanitis; psoriatic arthritis; synovitis; meningitis; encephalitis;
Bickerstaff's encephalitis; ncephalomyelitis; spondylitis; osteomyelitis; Guillain-barre syndrome; myelitis; neuromyelitis optica; cystitis; acute inflammation on an infected or wounded site; nephritis; and glomerulonephritis.

7. The pharmaceutical composition of claim 5, wherein the inflammatory disease is inflammatory disease of a digestive system.

8. A health functional food for preventing or ameliorating inflammatory disease, the health functional food comprising, as an active ingredient, a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing.

9. A health functional food for amelioration of intestinal health, the health functional food comprising, as an active ingredient, a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing.

10. The health functional food of claim 9, wherein the amelioration of intestinal health includes an aid to growth of beneficial intestinal bacteria and inhibition of harmful bacteria, an aid to intestinal health by immunity control, or an aid to smooth bowel movements.

11. A pharmaceutical composition for preventing or treating bacterial infection, the pharmaceutical composition comprising, as an active ingredient, a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing.

12. The pharmaceutical composition of claim 11, wherein the bacterial infection is infection caused by gram-negative bacteria.

13. The pharmaceutical composition of claim 12, wherein the infection caused by gram-negative bacteria is caused by bacteria belonging to the Clostridium sp., the Helicobacter sp., the Escherichia sp., or the Salmonella sp.

14. The pharmaceutical composition of claim 11, wherein the bacterial infection is Clostridium difficile infection (CDI) or Clostridium difficile associated diarrhea.

15. An antibacterial composition for external preparation on skin, the antibacterial composition comprising, as an active ingredient, a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing.

16. A health functional food for preventing or ameliorating bacterial infection, the health functional food comprising, as an active ingredient, a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing.

17. A method of preventing or treating inflammatory disease, the method comprising administering a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing, to a subject in need of the foregoing.

18. A method of preventing or treating bacterial infection, the method comprising administering a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing, to a subject in need of the foregoing.

19. Use of a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing, in preparation of a composition for preventing or treating inflammatory disease.

20. Use of a Blautia massiliensis strain, a Blautia obeum strain, a Blautia wexlerae strain, a Leuconostoc lactis strain, or a Ruminococcus bromii strain, a vesicle derived from the strains, a lysis solution of the strains, a culture solution of the strains, or a mixture of the foregoing, in preparation of a composition for preventing or treating bacterial infection.


SEQUENCE LISTING (5 sequences)

SEQ ID NO: 1 - DNA, 1343 bp, synthetic construct
  misc_feature 1..1343: note = 16S rRNA for Blautia massiliensis
  source 1..1343: mol_type = other DNA
cttcggcagatttagctggtttctagtggcggacgggtgagtaacgcgtgggtaacctgccttgtacagggggataacagtcagaaatgactgctaataccgcataagcgcacaggaccgcatggtccagtgtgaaaaactccggtggtataagatggacccgcgttggattagctggttggtggggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagaccgatggacaagtctgatgtgaaaggctggggctcaaccccgggactgcattggaaactgcccgtcttgagtgccggaaaggtaagcggaattcctagtgtagcggtgaaatgcgtagatattaagaggaacaccagtggcgaaggcggcttactggacggtaactgacgttgaggctcgaaagcgtggggagcaaacaggattagataccctggtagtccacgcggtaaacgatgaatgctaggtgtcgggtgacaaagttcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatccttctgaccggaacttaaccgttccttcccttcggggcagaagtgacaggtggtgcatggttgtcgtcagctcgtgtcgtgagatgttgggttaagtcccgcaacgagcgcaacccctatcctcagtagccagcagttcggctgggcactctgtggagactgccagggataacctggaggaaggcggggatgacgtcaaatcatcatgccccttatgatttgggctacacacgtgctacaatggcgtaaacaaagggaagcgaacctgcgagggtgggcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccggaaagtcag

SEQ ID NO: 2 - DNA, 1457 bp, synthetic construct
  misc_feature 1..1457: note = 16S rRNA for Blautia obeum
  source 1..1457: mol_type = other DNA
acgacttcaccccagtcatcggtcccgccttcggcagctccttctttgcagttaggtcactgacttcgggcgttactgactcccatggtgtgacgggcggtgtgtacaagacccgggaacgtattcaccgcggcattctgatccgcgattactagcgattccagcttcgtgcagtcgagttgcagactgcagtccgaactgggacgttatttttgggatttgcttaccttcgcaggcttgcttccctttgtttacgccattgtagcacgtgtgtagcccaaatcataaggggcatgatgatttgacgtcatccccgccttcctccaggttatccctggcagtctcctcagagtgcccagccggactgctggctactggggataggggttgcgctcgttgcgggacttaacccaacatctcacgacacgagctgacgacaaccatgcaccacctgtctcctctgtcccgaaggaaagttccggttaaggaacggtcagagggatgtcaagacttggtaaggttcttcgcgttgcttcgaattaaaccacatgctccaccgcttgtgcgggtccccgtcaattcctttgagtttcattcttgcgaacgtactccccaggtggaatacttaatgcgttgcggcggcaccgaagagctttgctccccaacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggcactcaagactaacagtttccaatgcagtccaggggttgagcccccgcctttcacatcagacttgccagtccgtctacgctccctttacacccagtaaatccggataacgcttgccccctacgtattaccgcggctgctggcacgtagttagccggggcttcttagtcaggtaccgtcactatcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgttcaccctctcaggccggctatggatcgtcgccttggtaggccgttaccctgccaactagctaatccaacgcgggtccatcttataccaccggagtttttcacaccggaccatgcggtcctgtgcgcttatgcggtattagcaaccatttctggttgttatccccctgtataaggcaggttacccacgcgttactcacccgtccgccactagaaaccaactaaatctgccgaagcttcaataaaaggtttcccgttcgacttgcaggtgttaagcacgccgccggcgttcat

SEQ ID NO: 3 - DNA, 1461 bp, synthetic construct
  misc_feature 1..1461: note = 16S rRNA for Blautia wexlerae
  source 1..1461: mol_type = other DNA
ttagacttcaccccagtcatcggtcccgccttcggcagctccttctttgcagttaggtcactgacttcgggcgttactgactcccatggtgtgacgggcggtgtgtacaagacccgggaacgtattcaccgcggcattctgatccgcgattactagcgattccagcttcgtgtagtcgggttgcagactacagtccgaactgggacgttatttttgggatttgctccatgtcaccatctcgcttccctttgtttacgccattgtagcacgtgtgtagcccaaatcataaggggcatgatgatttgacgtcatccccgccttcctccaggttatccctggcagtctccccagagtgcccaccttaaatgctggctactgaggataggggttgcgctcgttgcgggacttaacccaacatctcacgacacgagctgacgacaaccatgcaccacctgtctcctctgtcccgaaggaaagatccggttaaggatcggtcagagggatgtcaagacttggtaaggttcttcgcgttgcttcgaattaaaccacatgctccaccgcttgtgcgggtccccgtcaattcctttgagtttcattcttgcgaacgtactccccaggtggaatacttactgcgttgcgacggcaccgaagagctatgctccccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacccctccggcactcaagtatgacagtttccaatgcagtccacaggttgagcccatgcctttcacatcagacttgccacaccgtctacgctccctttacacccagtaaatccggataacgcttgccccctacgtattaccgcggctgctggcacgtagttagccggggcttcttagtcaggtaccgtcactatcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgttcaccctctcaggccggctatggatcgtcgccttggtgggccgttaccccaccaacaagctaatccaacgcgggtccatcttataccaccggagtttttcacactgagccatgcagctctgtgcgcttatgcggtattagcagccatttctgactgttatccccctgtataaggcaggttacccacgcgttactcacccgtccgccactagaattaaattaaatcgaccgaagtttcaataaagtaattcccgttcgacttgcatgtgttaagcacgccgcccgcgttcatccg

SEQ ID NO: 4 - DNA, 1491 bp, synthetic construct
  misc_feature 1..1491: note = 16S rRNA for Leuconostoc lactis
  source 1..1491: mol_type = other DNA
cctgttacgacttcaccctagtcatctgtcctgccttagacggctccctccataaggttaggccaccggctttgggcattacaaactcccatggtgtgacgggcggtgtgtacaagacccgggaacgtattcaccgcggcgtgctgatccgcgattactagcgattccgacttcgtgcagtcgagttgcagactgcagtccgaactgagacgtactttaagagattagctcaccctcgcgggttggcaactcgttgtatacgccattgtagcacgtgtgtagcccaggtcataaggggcatgatgatctgacgtcgtccccgccttcctccggtttgtcaccggcagtctcgctagagtgcccaactgaatgctggcaactaacaataagggttgcgctcgttgcgggacttaacccaacatctcacgacacgagctgacgacgaccatgcaccacctgtcactttgtctccgaagagaacacttctatctctagaagcttcaaaggatgtcaagacctggtaaggttcttcgcgttgcttcgaattaaaccacatgctccaccgcttgtgcgggtccccgtcaattcctttgagtttcaaccttgcggtcgtactccccaggcggaatacttaatgcgttagcttcggcactaagaggcggaaacctcctaacacctagtattcatcgtttacggtgtggactaccagggtatctaatcctgtttgctacccacactttcgagcctcaacgtcagttgttgtccagtaagccgccttcgccactggtgttcttccatatatctacgcattccaccgctacacatggagttccacttacctctacaacactcaagttaaccagtttccaatgccattccggagttgagctccgggctttcacatcagacttaatcaaccgtctgcgctcgctttacgcccaataaatccggataacgctcgggacatacgtattaccgcggctgctggcacgtatttagccgtccctttctggtatggtaccgtcgaactagaatcattccctattctagcatttcttcccatacaacagtgctttacgaccctaaagccttcatcacacacgcggcgttgctccatcaggctttcgcccattgtggaagattccctactgcagcctcccgtaggagtttgggccgtgtctcagtcccaatgtggccgatcagtctctcaactcggctatgcatcattgtcttggtaggcctttaccccaccaactaactaatgcaccgcggacccatctctaggtgacgccgtagcgcctttcaactttgtatcatgcgatactaagttttattcggtattagcatctgtttccaaatgttatccccagccttgaggcaggttatccacgtgttactcacccgttcgccactcgcttgaaaggtgcaagcacctttcgctgcgcgttcgacttgcatgtattaggcacgccgccagcgttcatcctgagc

SEQ ID NO: 5 - DNA, 1456 bp, synthetic construct
  misc_feature 1..1456: note = 16S rRNA for Ruminococcus bromii
  source 1..1456: mol_type = other DNA
gacttcaccccagtcgccaatcctaccttcggcagcgtcctcacgaggttggactactgacttcgggtattaccggctcccatggtgtgacgggcggtgtgtacaaggcccgggaacgtattcaccgcggcatgctgatccgcgattactagcaattccatcttcatgcaggcgggttgcagcctgcaatctgaactgagaatgtttttaggggtttgctccacatcgctgtcttgcttccctctgttaacatccattgtagtacgtgtgtagcccaggtcataaggggcatgatgatttgacgtcgtccccaccttcctccgttttgtcaacggcagtcctattagagtgctcttgcgtagcaactaatagcaagggttgcgctcgttgcgggacttaacccaacatctcacgacacgagctgacgacaaccatgcaccacctgtctcaactttccccgaagggcacctaatgtatctctacttcgttagttggatgtcaagacctggtaaggttcttcgcgttgcttcgaattaaaccacatactccactgcttgtgcgggcccccgtcaattcctttgagtttcaaccttgcggccgtactccccaggtggattacttattgtgttaactccgacacggaaggggtcagaccccccacacctagtaatcatcgtttacagcgtggactaccagggtatctaatcctgtttgctacccacgctttcgtgcctcagcgtcagttaaagcccagcaggccgccttcgccactggtgttcctccccatctctacgcatttcaccgctacacggggaattccgcctgcctctacttcactcaagctctacagtttcaaatgcagtttatgggttgagcccatagatttcacatctgacttgcaaagccgcctacgcaccctttacacccagtaaatccggacaacgcttgctccctacgtattaccgcggctgctggcacgtagttagccggagcttattcattaagtaccgtcaagtttcgtccttaataaaagaagtttacaatccgaaaaccttcatccttcacgcggcgttgctgcgtcagggttgcccccattgcgcaatatcccccactgctgcctcccgtaggagtctgggccgtgtctcagtcccaatgtggccgttcaacctctcagtccggctaccgatcgcggtcttggtgagccgttacctcaccaactaactaatcggacgcgagtccatcttcaagcgataaaatctttgatatcaaaaccatgtggttccgatatgtcatgcggtattagcattcttttcagaatgttatccccctcttgaaggcaggttactcacgcgttactcacccgtccgccactaaactaaatcaaattcattccgaagaaatctttcaaaacagttccgttcgacttgcatgtgttaggcacgccgccggcgttcgt